(19) United States
(12) Patent Application Publication
(10) Pub. No.: US 2025/0275966 A1
(43) Pub. Date: Sep. 4, 2025

(54) GABAA RECEPTOR MODULATOR SALTS, PARTICLES, AND USES THEREOF

(71) Applicant: Engrail Therapeutics, Inc., San Diego, CA (US)

(72) Inventors: Vikram Sudarsan, San Diego, CA (US); David Garvey, Dover, MA (US); Eve Taylor, Descanso, CA (US); Sunil Mhaskar, Foster City, CA (US); William Brubaker, Cheshire, CT (US)

(21) Appl. No.: 18/858,125

(22) PCT Filed: Apr. 20, 2023
(86) PCT No.: PCT/US2023/065994

§ 371 Date: Oct. 18, 2024

Related U.S. Application Data
(60) Provisional application No. 63/333,075, filed on Apr. 20, 2022.

(51) Int. Cl. A61K 31/5025; C07C 57/15; C07D 487/04

(52) U.S. Cl. CPC A61K 31/5025; C07C 57/15; C07D 487/04

(57) ABSTRACT

Described herein are salts (e.g., fumarate salts) of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine and particles comprising 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine or a salt thereof, and uses thereof.

### RELATED APPLICATIONS

This application claims priority of U.S. Provisional Patent Application No. 63,333,075, filed Apr. 20, 2023, the entire content of which is incorporated herein by reference.

### BACKGROUND

GABA receptors respond to the neurotransmitter gamma-aminobutyric acid (GABA), which is the major inhibitory compound of the vertebrate central nervous system. GABA_{A} receptors occur in all organisms that have a nervous system. Modulation of GABA_{A} receptors may therefore be useful in therapeutically addressing diseases or disorders of the central nervous system. 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine (Compound 1) is a GABA_{A} receptor modulator, but has limited bioavailability and a short half-life in mice. Accordingly, there remains a need for GABA_{A} receptor modulator therapeutics, including improved forms of Compound 1.

### SUMMARY

Described herein are salts of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine and uses thereof, as well as particles comprising 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine or a salt thereof and uses thereof.

Compound 1 is shown below as a free base (wherein D is deuterium).

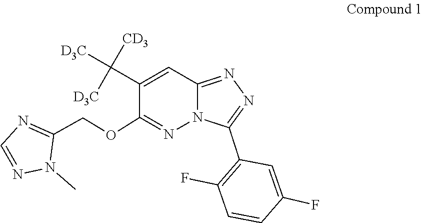 [SMILES: [2H]C([2H])([2H])C(c1cc2nnc(-c3cc(F)ccc3F)n2nc1OCc1ncnn1C)(C([2H])([2H])[2H])C([2H])([2H])[2H]]

3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine

### BRIEF DESCRIPTION OF THE DRAWINGS

FIG. 1 shows a particle size distribution plot corresponding to a particle batch generated in Example 1 (particle batch 1).

FIG. 2 shows a particle size distribution plot corresponding to a comparator particle batch (particle batch 2a) generated in Example 2.

FIG. 3 shows a particle size distribution plot corresponding to another comparator particle batch (particle batch 2b) generated in Example 2.

FIG. 4 shows an XRPD (X-Ray Powder Diffraction) trace of form A hemi-fumarate salt of Compound 1.

FIG. 5 shows an XRPD trace of form B hemi-fumarate salt of Compound 1.

FIG. 6 shows DSC (Differential Scanning Calorimetry) and TGA (Thermogravimetric Analysis) traces of form A hemi-fumarate salt of Compound 1.

FIG. 7 shows DSC and TGA traces of form B hemi-fumarate salt of Compound 1.

FIG. 8 shows an XRPD trace of form A sulfate salt of Compound 1.

FIG. 9 shows an XRPD trace of form A hydrochloride salt of Compound 1.

FIG. 10 shows an XRPD trace of form A phosphate salt of Compound 1.

FIG. 11 shows an XRPD trace of form B phosphate salt of Compound 1.

FIG. 12 shows an XRPD trace of form A tosylate salt of Compound 1.

FIG. 13 shows an XRPD trace of form A malonate salt of Compound 1.

FIG. 14 shows an XRPD trace of form A maleate salt of Compound 1.

### DETAILED DESCRIPTION

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.”

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The terms “composition” and “pharmaceutical composition” refer to a mixture of at least one compound described herein with a carrier or a pharmaceutically acceptable carrier, respectively. The pharmaceutical composition facilitates administration of the compound to a patient or subject. Multiple techniques of administering a composition exist including, but not limited to, intravenous, oral, nasal, rectal, intravaginal, aerosol, parenteral, buccal, sublingual, ophthalmic, pulmonary, transdermal and topical administration.

The terms “effective amount” and “therapeutically effective amount” refer to an amount of therapeutic compound, such as a compound described herein, administered to a subject, either as a single dose or as part of a series of doses, which is effective to produce a desired therapeutic effect.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its salt form. Lists of salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

A “signal” may present as a component of a broadened peak, a shouldered peak, or a split peak, which result from two or more overlapping or adjacent signals.

The term “solid form” includes, but is not limited to, polymorphs, crystalline forms, amorphous forms, solvates, and hydrates of a compound.

The terms “substituted” or “substitution” refers to replacement of hydrogen attached to another group with an atom or group of atoms as the replacement substituent, wherein each substituent is independently selected.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability.

References have been made to patents and printed publications throughout this specification, each of which are individually incorporated herein by reference in their entirety.

It is to be understood that the embodiments of this disclosure are illustrative. Accordingly, the present disclosure is not limited to that precisely as shown and described.

### Compounds and Solid Forms

3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine (Compound 1) is a GABA_{A} receptor modulator that can act at the benzodiazepine site of the GABA_{A} receptor as a selective allosteric modulator of the α2, α3, and α5 subtypes.

Compound 1 and its salts as described herein are synthesized using any suitable procedures starting from compounds that are available from commercial sources, or are prepared using procedures described herein. General methods for the preparation of a compound as described herein are modified by the use of appropriate reagents and conditions, for the introduction of the various moieties found in the formula as provided herein.

In some embodiments, Compound 1 may be prepared using the methods described in U.S. Pat. Nos. 8,003,646, 8,399,467, or 8,921,366, the entire content of each of which are incorporated by reference. The preparation of a compound corresponding to a non-deuterated form of Compound 1 is described in the Journal of Medicinal Chemistry, 48 (23): 7089-92 (Carling et al., “7-(1,1-Dimethylethyl)-6-(2-ethyl-2H-1,2,4-triazol-3-ylmethoxy)-3-(2-fluorophenyl)-1,2,4-triazolo[4,3-b]pyridazine: a functionally selective gamma-aminobutyric acid(A) (GABA(A)) alpha2/alpha3-subtype selective agonist that exhibits potent anxiolytic activity but is not sedating in animal models”). Thus, Compound 1 may be prepared in a similar manner as Carling et al. by substitution with the appropriate corresponding deuterated reagents.

Preparation of a salt of Compound 1 may occur by, for example, contacting Compound 1 with an acid in a solvent solution, and isolating the salt of Compound 1 by removing the solvent.

Thus, in some embodiments, provided herein are compounds which are fumarate salts of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine.

In some embodiments, the compound is a hemi-fumarate salt of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine.

In some embodiments, the compound is of the formula:

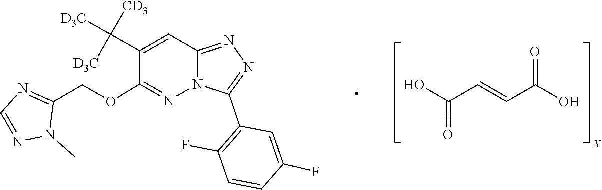

wherein X is 0.5, 1, or 2.

In some embodiments, provided herein are compounds which are sulfate salts of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine. In some embodiments, the sulfate salt of Compound 1 is prepared by combining 880 mg (2.15 mmol) of Compound 1 with 34.2 mL acetone. To the resulting solution (with minimal residual solids), sulfuric acid (2.5 M in water, 861 μL, 1.0 eq) was added. The resulting slurry was seeded with crystalline Form A (˜10 mg). The slurry was heated to 40° C. (did not dissolve), cooled to 20° C. at 0.1° C./min (held for 1 h at every 2° C. interval) and stirred at 20° C. overnight. The solids were isolated by vacuum filtration and air-dried overnight. The yield was 83% (905 mg, 1.79 mmol) of Form A sulfate salt of Compound 1.

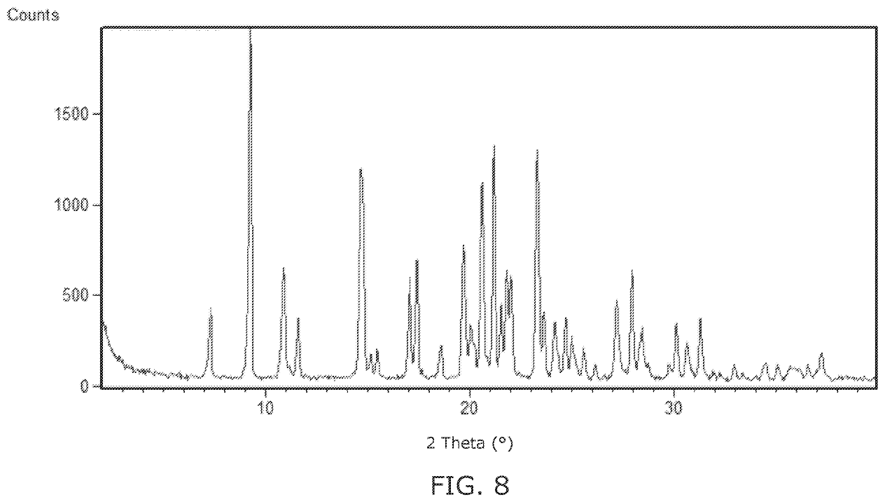

In some embodiments, the sulfate salt of Compound 1 has an XRPD pattern substantially as shown in FIG. 8. In some embodiments, the sulfate salt of Compound 1 has at least one signal, in terms of 20, selected from Table A.

TABLE A
Form A sulfate salt of Compound 1 XRPD signals.

| 2Θ [cts] | Height [cts] | d-spacing [Å] | Rel. Int. [%] |
|---|---|---|---|
| 7.29 | 253 | 12.1 | 19 |
| 9.25 | 1331 | 9.55 | 100 |
| 10.87 | 408 | 8.13 | 31 |
| 11.59 | 221 | 7.63 | 17 |
| 14.65* | 810 | 6.04 | 61 |
| 14.77* | 737 | 5.99 | 55 |
| 17.04 | 399 | 5.20 | 30 |
| 17.39 | 492 | 5.10 | 37 |
| 19.68 | 539 | 4.51 | 41 |
| 20.58 | 805 | 4.31 | 61 |
| 21.17 | 945 | 4.19 | 71 |
| 21.79 | 441 | 4.08 | 33 |
| 22.01 | 418 | 4.03 | 31 |
| 23.29 | 949 | 3.82 | 71 |
| 27.17 | 325 | 3.28 | 24 |
| 27.92 | 460 | 3.19 | 35 |
| 30.09 | 250 | 2.97 | 19 |
| 30.62 | 152 | 2.92 | 11 |
| 31.27 | 260 | 2.86 | 20 |
| 32.92 | 61 | 2.72 | 5 |

In some embodiments, provided herein are compounds which are hydrochloride salts of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine. In some embodiments, the hydrochloride salt of Compound 1 is prepared by combining 1.08 g, (2.65 mmol) of Compound 1 with acetonitrile (36.8 mL, 34 vol). To the resulting solution (with minimal residual solids), hydrochloric acid (3.0 M in water, 883 μL, 1.0 eq) was added. The obtained slurry was seeded with crystalline Form A (˜10 mg). The slurry was heated to 40° C. (did not dissolve). The slurry was stirred at 40° C. for 2 h, cooled to 20° C. at 0.1° C./min rate and held for 1 h at every 2° C. interval and stirred at 20° C. overnight. The solids were isolated by vacuum filtration, air-dried for 3 h. The yield was 77% (0.912 g, 2.05 mmol) of Form A hydrochloride salt of Compound 1.

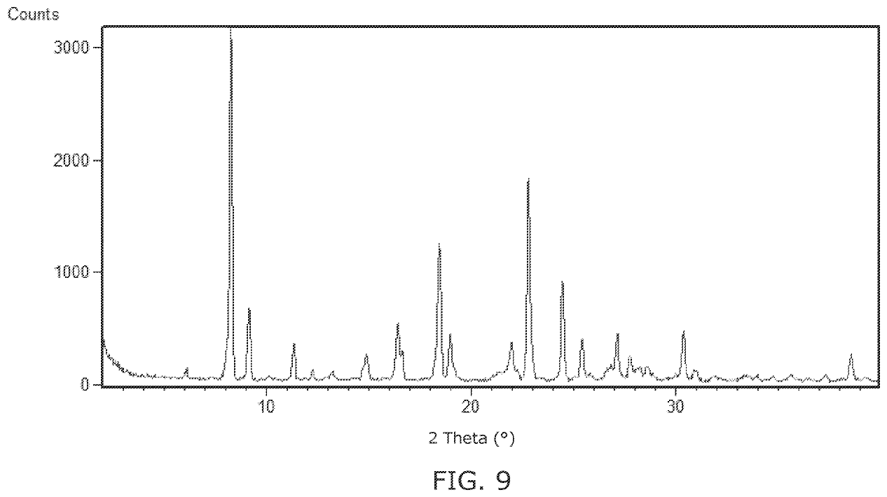

In some embodiments, the hydrochloride salt of Compound 1 has an XRPD pattern substantially as shown in FIG. 9. In some embodiments, the hydrochloride salt of Compound 1 has at least one signal, in terms of 20, selected from Table B.

TABLE B
Form A hydrochloride salt of Compound 1 XRPD signals.

| 2Θ [cts] | Height [cts] | d-spacing [Å] | Rel. Int. [%] |
|---|---|---|---|
| 8.27 | 2189 | 10.7 | 100 |
| 9.14 | 450 | 9.67 | 21 |
| 11.32 | 225 | 7.81 | 10 |
| 12.24 | 60 | 7.23 | 3 |
| 14.86 | 144 | 5.96 | 7 |
| 16.41 | 338 | 5.40 | 15 |
| 18.43 | 891 | 4.81 | 41 |
| 18.96 | 296 | 4.68 | 14 |
| 21.94 | 190 | 4.05 | 9 |
| 22.79 | 1186 | 3.90 | 54 |
| 24.44 | 668 | 3.64 | 31 |
| 25.38 | 289 | 3.51 | 13 |
| 27.09 | 423 | 3.29 | 19 |
| 27.73 | 229 | 3.21 | 10 |
| 30.34 | 330 | 2.94 | 15 |
| 38.52 | 194 | 2.33 | 9 |

In some embodiments, provided herein are compounds which are phosphate salts of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine. In some embodiments, the phosphate salt of Compound 1 is prepared by combining 1.04 g (2.53 mmol) with acetone (34.2 mL, 33 vol). To the resulting solution (with minimal residual solids) phosphoric acid (3.0 M in water, 845 μL, 1.0 eq) was added. The obtained slurry was seeded with crystalline Form A (˜10 mg). The slurry was heated to 40° C. (did not dissolve). The slurry was stirred at 40° C. for 2 h, cooled to 20° C. at 0.1° C./min rate and held for 1 h at every 2° C. interval and stirred at 20° C. overnight. The solids were isolated by vacuum filtration, air-dried for 4 h. The yield was 84% (1.07 g, 2.12 mmol) of Form A phosphate salt of Compound 1.

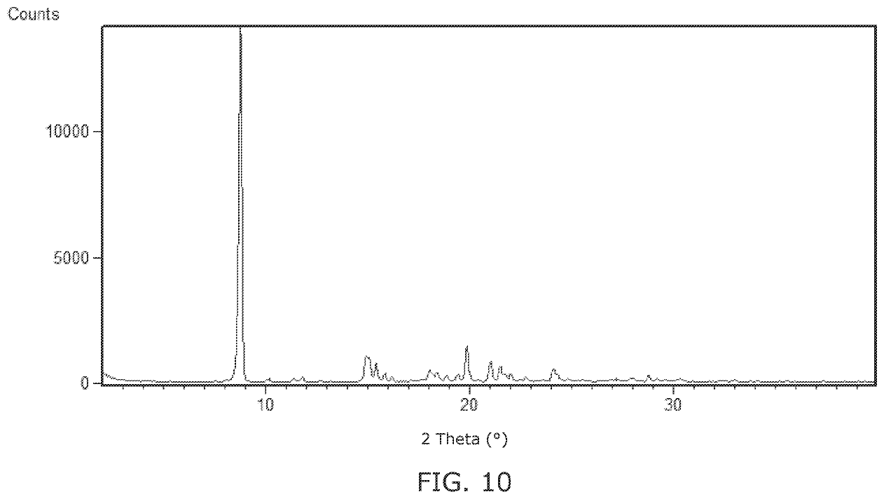

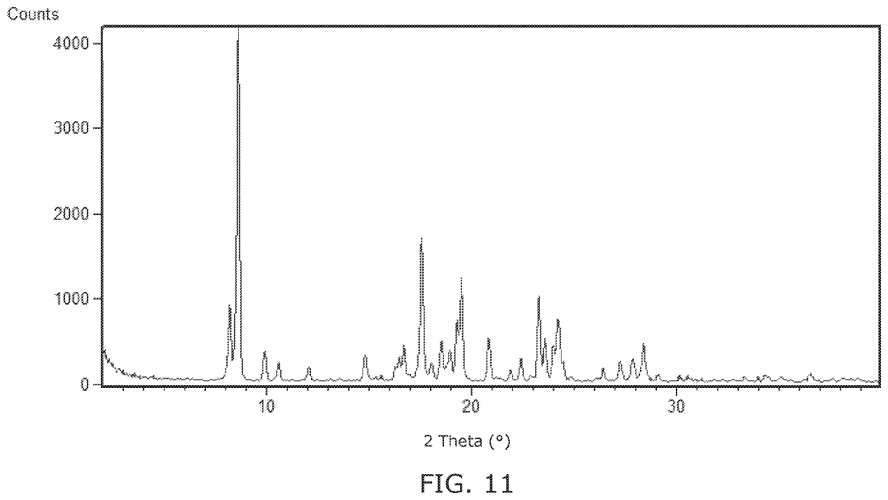

In some embodiments, the phosphate salt of Compound 1 has an XRPD pattern substantially as shown in FIG. 10 or FIG. 11. In some embodiments, the phosphate salt of Compound 1 has at least one signal, in terms of 20, selected from Table C or Table D.

TABLE C
Form A phosphate salt of Compound 1 XRPD signals.

| 2Θ [cts] | Height [cts] | d-spacing [Å] | Rel. Int. [%] |
|---|---|---|---|
| 8.76 | 9711 | 10.1 | 100 |
| 10.13 | 102 | 8.72 | 1 |
| 11.35 | 113 | 7.79 | 1 |
| 11.78 | 154 | 7.50 | 2 |
| 14.91 | 710 | 5.94 | 7 |
| 15.08 | 704 | 5.87 | 7 |
| 15.41 | 531 | 5.75 | 5 |
| 15.82 | 253 | 5.60 | 3 |
| 18.04 | 282 | 4.91 | 3 |
| 18.40 | 244 | 4.82 | 3 |
| 18.85 | 189 | 4.70 | 2 |
| 19.40 | 215 | 4.57 | 2 |
| 19.84 | 1035 | 4.47 | 11 |
| 20.99 | 605 | 4.23 | 6 |
| 21.50 | 372 | 4.13 | 4 |
| 22.00 | 237 | 4.04 | 2 |
| 24.13 | 290 | 3.69 | 3 |

TABLE D
Form B phosphate salt of Compound 1 XRPD signals.

| 2Θ [cts] | Height [cts] | d-spacing [Å] | Rel. Int. [%] |
|---|---|---|---|
| 8.20 | 600 | 10.8 | 21 |
| 8.62 | 2877 | 10.3 | 100 |
| 9.91 | 238 | 8.91 | 8 |
| 10.59 | 141 | 8.35 | 5 |
| 12.05 | 117 | 7.34 | 4 |
| 14.81 | 220 | 5.98 | 8 |
| 17.55 | 1226 | 5.05 | 43 |
| 18.51 | 345 | 4.79 | 12 |
| 19.28 | 505 | 4.60 | 18 |
| 19.49 | 869 | 4.55 | 30 |
| 20.83 | 381 | 4.26 | 13 |
| 23.27 | 786 | 3.82 | 27 |
| 24.20 | 536 | 3.67 | 19 |
| 27.24 | 180 | 3.27 | 6 |
| 27.84 | 197 | 3.20 | 7 |
| 28.36 | 314 | 3.14 | 11 |

In some embodiments, provided herein are compounds which are tosylate salts of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine. In some embodiments, the tosylate salt of Compound 1 is prepared by combining 888 mg (2.17 mmol) with IPA (3.5 mL, 39 vol). To the resulting solution (with minimal residual solids), tosic acid (3.0 M in water, 725 μL, 1.0 eq) was added. The obtained thin slurry was heated to 40° C. for 2 h with stirring, becoming thicker gradually, and seeded with crystalline Form A (˜10 mg). The slurry was cooled to 20° C. at 0.1° C./min rate and held for 1 h at every 2° C. interval and stirred at 20° C. over the weekend. The solids were isolated by vacuum filtration, air-dried for 2 h. The yield was 87% (1.10 g, 1.89 mmol) of Form A tosylate salt of Compound 1.

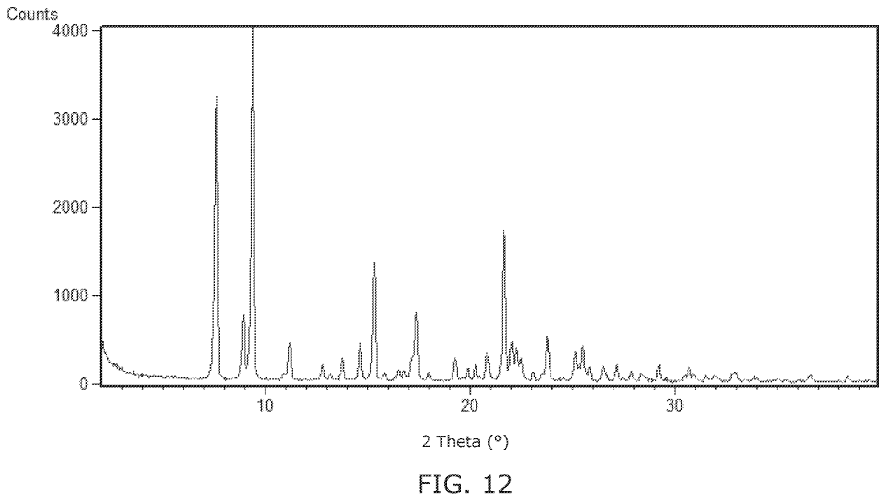

In some embodiments, the tosylate salt of Compound 1 has an XRPD pattern substantially as shown in FIG. 12. In some embodiments, the tosylate salt of Compound 1 has at least one signal, in terms of 20, selected from Table E.

TABLE E
Form A tosylate salt of Compound 1 XRPD signals.

| 2Θ [cts] | Height [cts] | d-spacing [Å] | Rel. Int. [%] |
|---|---|---|---|
| 7.59 | 2125 | 11.6 | 77 |
| 8.92 | 503 | 9.91 | 18 |
| 9.37 | 2755 | 9.43 | 100 |
| 11.18 | 309 | 7.91 | 11 |
| 12.79 | 121 | 6.92 | 4 |
| 13.75 | 170 | 6.44 | 6 |
| 14.62 | 293 | 6.06 | 11 |
| 15.30 | 1010 | 5.79 | 37 |
| 17.35 | 588 | 5.11 | 21 |
| 19.25 | 187 | 4.61 | 7 |
| 20.81 | 226 | 4.26 | 8 |
| 21.64 | 1280 | 4.10 | 46 |
| 22.02 | 322 | 4.03 | 12 |
| 22.24 | 273 | 3.99 | 10 |
| 22.46 | 179 | 3.96 | 7 |
| 23.76 | 371 | 3.74 | 13 |
| 25.12 | 238 | 3.54 | 9 |
| 25.47 | 289 | 3.49 | 10 |
| 26.51 | 120 | 3.36 | 4 |
| 27.13 | 140 | 3.28 | 5 |

In some embodiments, provided herein are compounds which are malonate salts of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine. In some embodiments, the malonate salt of Compound 1 is prepared by combining 1.09 g (2.68 mmol) with toluene (3.5 mL, 32 vol). To the resulting solution (with minimal residual solids), malonic acid (3.0 M in water, 893 μL, 1.0 eq) was added. The obtained thin slurry was heated to 40° C. for 2 h with stirring and seeded with crystalline Form A (˜10 mg). The slurry was cooled to 20° C. at 0.1° C./min rate and held for 1 h at every 2° C. interval and stirred at 20° C. over several days. The solids were isolated by vacuum filtration, air-dried for 2 h. The yield was 73% (0.996 g, 1.94 mmol) of Form A malonate salt of Compound 1.

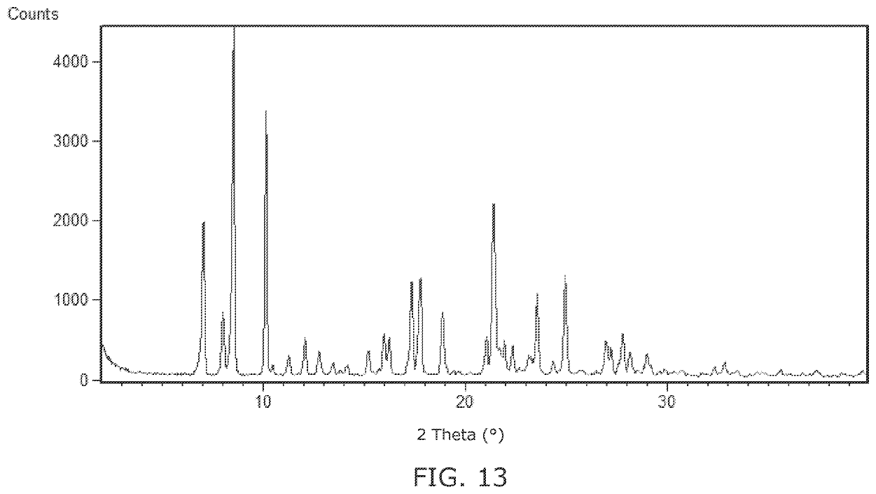

In some embodiments, the malonate salt of Compound 1 has an XRPD pattern substantially as shown in FIG. 13. In some embodiments, the malonate salt of Compound 1 has at least one signal, in terms of 20, selected from Table F.

TABLE F
Form A malonate salt of Compound 1 XRPD signals.

| 2Θ [cts] | Height [cts] | d-spacing [Å] | Rel. Int. [%] |
|---|---|---|---|
| 7.03 | 1299 | 12.6 | 43 |
| 8.00 | 559 | 11.0 | 18 |
| 8.53 | 3039 | 10.4 | 100 |
| 10.12 | 2233 | 8.73 | 73 |
| 11.25 | 172 | 7.86 | 6 |
| 12.06 | 323 | 7.33 | 11 |
| 12.77 | 197 | 6.93 | 6 |
| 15.20 | 227 | 5.83 | 7 |
| 15.98 | 357 | 5.54 | 12 |
| 16.22 | 339 | 5.46 | 11 |
| 17.32 | 838 | 5.11 | 28 |
| 17.76 | 868 | 4.99 | 29 |
| 18.86 | 555 | 4.70 | 18 |
| 21.03 | 375 | 4.22 | 12 |
| 21.38 | 1632 | 4.15 | 54 |
| 23.54 | 798 | 3.78 | 26 |
| 24.93 | 989 | 3.57 | 33 |
| 26.96 | 323 | 3.30 | 11 |
| 27.77 | 411 | 3.21 | 14 |
| 28.14 | 239 | 3.2 | 8 |

In some embodiments, provided herein are compounds which are maleate salts of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine. In some embodiments, the maleate salt of Compound 1 is prepared by combining 1.09 g (2.67 mmol) with toluene (3.5 mL, 32 vol). To the resulting solution (with minimal residual solids), maleic acid (3.0 M in water, 891 μL, 1.0 eq) was added. The obtained thin slurry was seeded with crystalline Form A (˜10 mg). The slurry was heated to 40° C. (did not dissolve). The slurry was stirred at 40° C. for 2 h, cooled to 20° C. at 0.1° C./min rate and held for 1 h at every 2° C. interval and stirred at 20° C. for several days. The solids were isolated by vacuum filtration, air-dried for 4 h. The yield was 91% (1.28 g, 2.43 mmol) of Form A maleate salt of Compound 1.

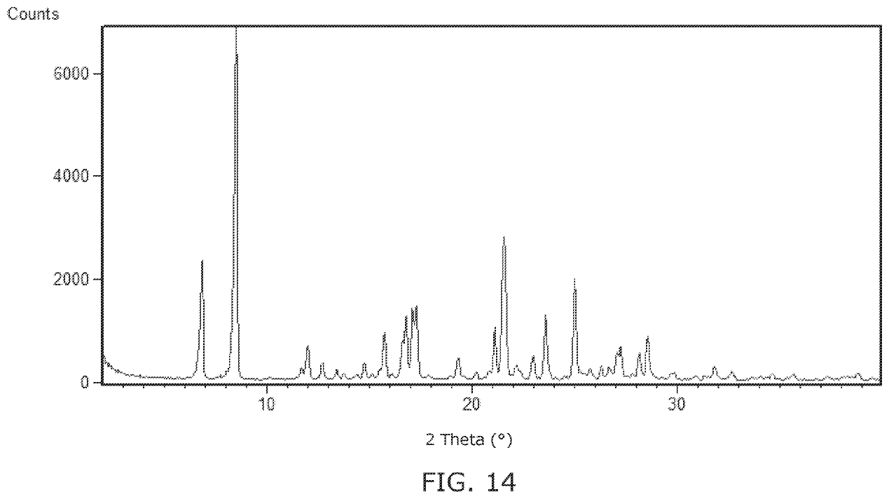

In some embodiments, the maleate salt of Compound 1 has an XRPD pattern substantially as shown in FIG. 14. In some embodiments, the maleate salt of Compound 1 has at least one signal, in terms of 2θ, selected from Table G.

TABLE G
Form A maleate salt of Compound 1 XRPD signals.

| 2Θ [cts] | Height [cts] | d-spacing [Å] | Rel. Int. [%] |
|---|---|---|---|
| 6.83 | 1552 | 12.9 | 34 |
| 8.48 | 4624 | 10.4 | 100 |
| 11.99 | 468 | 7.38 | 10 |
| 12.70 | 236 | 6.97 | 5 |
| 14.75 | 222 | 6.00 | 5 |
| 15.72 | 658 | 5.63 | 14 |
| 16.78 | 882 | 5.28 | 19 |
| 17.09 | 1022 | 5.18 | 22 |
| 17.28 | 1003 | 5.13 | 22 |
| 19.31 | 316 | 4.59 | 7 |
| 21.09 | 745 | 4.21 | 16 |
| 21.54 | 2131 | 4.12 | 46 |
| 22.95 | 346 | 3.87 | 7 |
| 23.56 | 964 | 3.77 | 21 |
| 24.98 | 1569 | 3.56 | 34 |
| 27.04 | 381 | 3.29 | 8 |
| 27.22 | 478 | 3.27 | 10 |
| 28.12 | 367 | 3.17 | 8 |
| 28.53 | 653 | 3.13 | 14 |

In some embodiments, the compound is an anhydrate, hemihydrate, monohydrate, or dihydrate. In some embodiments, the compound is a solvate. In some embodiments, the compound is in a solid form.

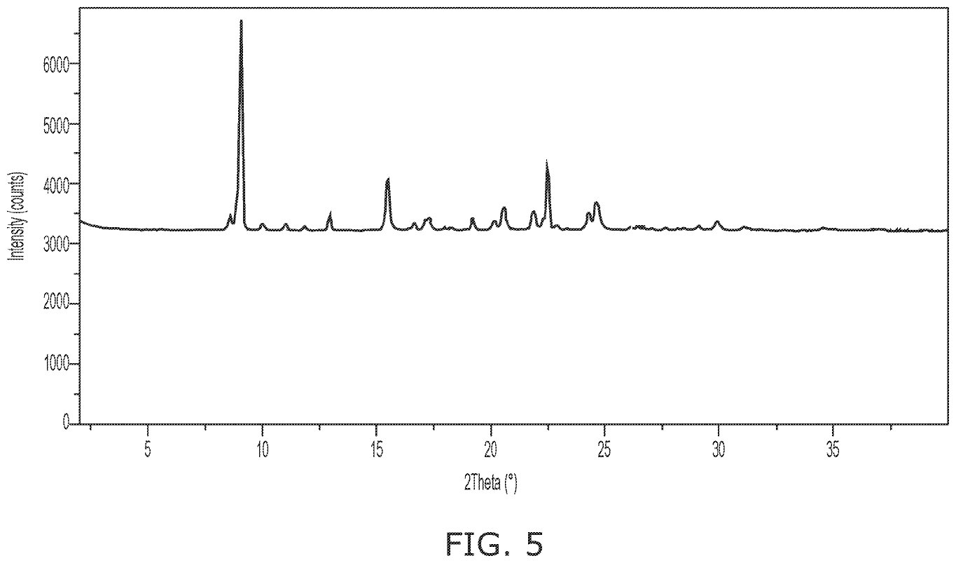

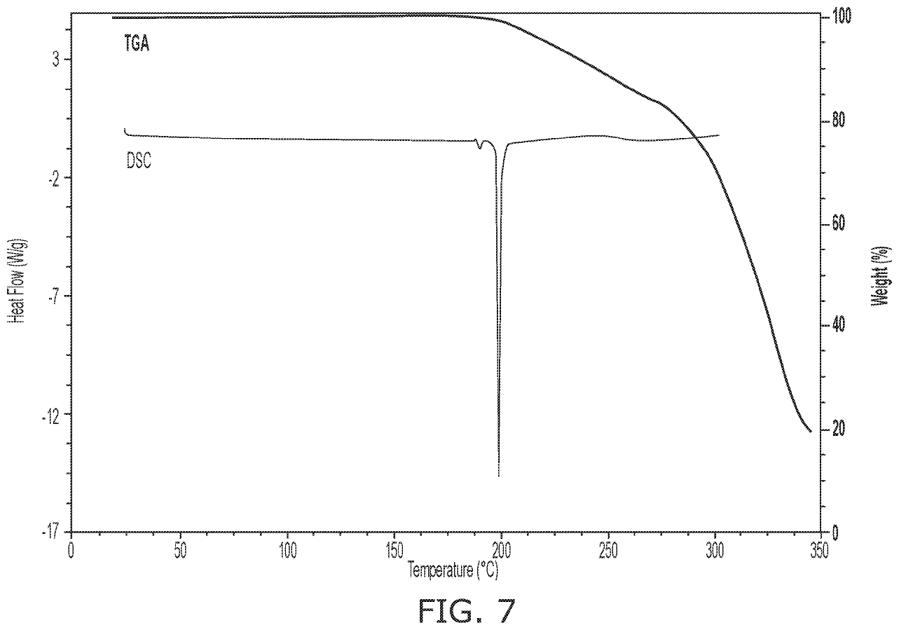

In some embodiments, the solid form B hemi-fumarate salt has an X-ray powder diffraction pattern comprising a 2-theta signal, based on CuKα1 radiation (1.54060 Å), at about 9.05+0.2°. In some embodiments, the solid form B hemi-fumarate salt has an X-ray powder diffraction pattern comprising 2-theta signals, based on CuKα1 radiation (1.54060 Å), at about 9.05+0.2°, about 15.46+0.2°, about 22.55+0.2°, and about 24.61+0.2°. In some embodiments, the solid form B hemi-fumarate salt has an X-ray powder diffraction pattern substantially as shown in FIG. 5. In some embodiments, the solid form B hemi-fumarate salt has a differential scanning calorimetry thermogram comprising an endothermic signal at about 199.6±3.0 (e.g., ±0.5)° C. In some embodiments, the solid form B hemi-fumarate salt has a differential scanning calorimetry thermogram substantially as shown in FIG. 7. In some embodiments, the solid form B hemi-fumarate salt has a thermogravimetric analysis substantially as shown in FIG. 7.

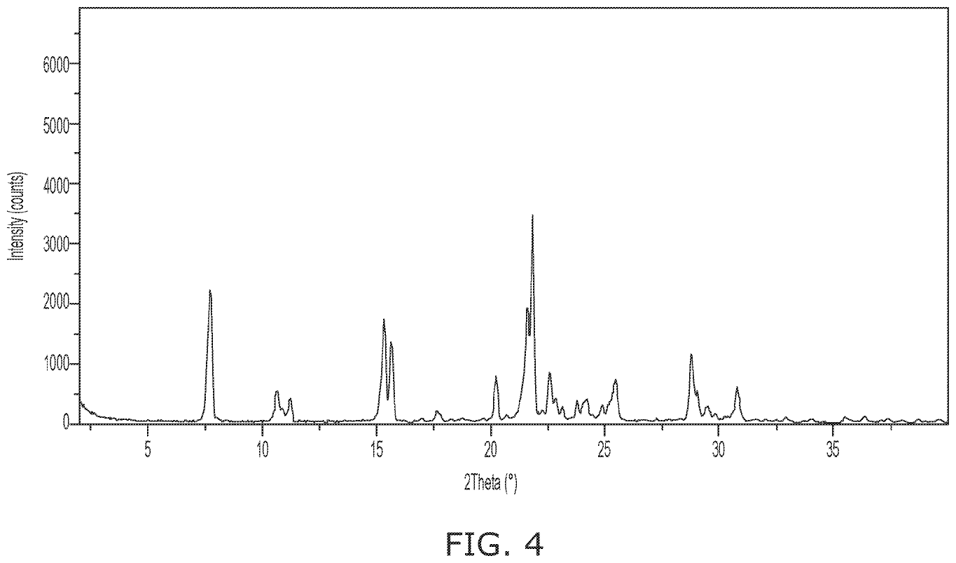

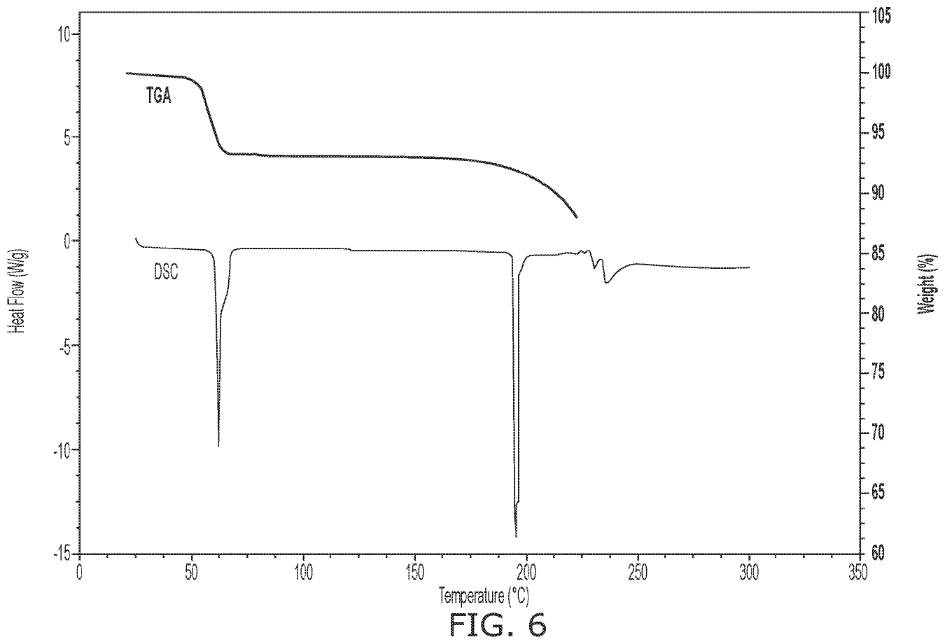

In some embodiments, the solid form A hemi-fumarate salt has an X-ray powder diffraction pattern comprising 2-theta signals, based on CuKα1 radiation (1.54060 Å), at about 7.70±0.2° or about 21.80±0.2°. In some embodiments, the solid form A hemi-fumarate salt has an X-ray powder diffraction pattern comprising 2-theta signals, based on CuKα1 radiation (1.54060 Å), at about 7.70±0.2°, about 21.80±0.2°, about 25.44±0.2°, and about 28.75±0.2°. In some embodiments, the solid form A hemi-fumarate salt has an X-ray powder diffraction pattern substantially as shown in FIG. 4. In some embodiments, the solid form A hemi-fumarate salt has a differential scanning calorimetry thermogram comprising an endothermic signal at about 62.1±3.0 (e.g., +0.5)° C., about 195.2±3.0 (e.g., +0.5)° C., or both. In some embodiments, the solid form A hemi-fumarate salt has a differential scanning calorimetry thermogram substantially as shown in FIG. 6. In some embodiments, the solid form A hemi-fumarate salt has a thermogravimetric analysis substantially as shown in FIG. 6.

Powders analyzed by XRPD spectroscopy may include components other than the crystalline compound meant to be identified, which may result in signals present in an XRPD diffractogram in addition to those attributed to the crystalline compound to be identified. A particular compound may also include two or more adjacent or overlapping signals. Thus, in some embodiments, an XRPD signal may present as a component of a broadened peak, a shouldered peak, or a split peak, which result from two or more overlapping or adjacent signals. In some embodiments, an XRPD signal may be synonymous with an XRPD peak.

In some embodiments, the compound or solid form is substantially purified. In some embodiments, the compound or solid form is crystalline. In some embodiments, the compound or solid form is prepared by a process comprising precipitating the compound from a solution comprising 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine, fumaric acid, and a solvent comprising acetone or acetonitrile. In some embodiments, the process comprises drying the precipitated compound. In some embodiments, the precipitated compound, which may be crystalline, is reduced (e.g., ground, collided, or tumbled) to particles having one or more of the particle characteristics described below.

In some embodiments, compositions are provided comprising one or more compounds described herein. In some embodiments, the compositions are pharmaceutical compositions.

The compositions may further comprise a pharmaceutically acceptable carrier.

In some embodiments, the compound is present in the composition in an amount of at least about 90% by weight.

In some embodiments, the composition is a pharmaceutical composition consisting essentially of the compound.

### Particles

In some embodiments, described herein are particles, comprising a compound of the formula:

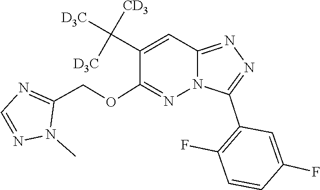 [SMILES: [2H]C([2H])([2H])C(c1cc2nnc(-c3cc(F)ccc3F)n2nc1OCc1ncnn1C)(C([2H])([2H])[2H])C([2H])([2H])[2H]]

or a salt thereof.

In some embodiments, the compound is about, or at least about, 75, 80, 85, 90, 95, or 100% by mass of the particle.

In some embodiments, the particle comprises a particle surface wherein the particle surface comprises a coating on at least a portion of the particle surface. In some embodiments, the coating comprises a film coating. In some embodiments, the particle comprises a film coating with a polymer or co-polymer to form microcapsules, which may be used to form chewable taste-masked granules. In some embodiments, the coating comprises a polymer or co-polymer. In some embodiments, the coating comprises one or more of cellulose acetate phthalate, cellulose acetate trimellate, ethyl cellulose, glycol, hydroxy propyl cellulose, hydroxy propyl methyl cellulose, hydroxy propyl methyl cellulose phthalate, methacrylic acid co-polymer, high molecular weight polyethylene, polyvinyl alcohol, polyvinyl pyrrolidone, starch, or shellac. In some embodiments, the coating comprises a varnish (e.g., a non-nutritive varnish). In some embodiments, the coating comprises a sugar. In some embodiments, the coating comprises a sugar coating. In some embodiments, the particles described herein are sugar coated. In some embodiments, the particles described herein are not sugar coated.

In some embodiments, a dosage form comprising a plurality of particles comprises coated particles wherein the coating is selected, independently for each particle, from a coating described herein. Accordingly, in some embodiments a plurality of particles may include a mixture of enteric coated particles and extended release coated particles.

In some embodiments, the particles described herein are encapsulated within a coating.

In some embodiments of the particles described herein, the coating is 25% or less by mass of the coated particle.

In some embodiments, the particle comprises a diameter of about 0.2-20 μm (e.g., about 1-10 μm, e.g., about 2-6 μm, e.g., about 4 μm).

In some embodiments, the particles described herein are provided as a composition, comprising a plurality of particles, which may include one or more carriers. In some embodiments, the plurality of particles is encapsulated in a capsule, a compression coating, a film coating, or a powder coating. In some embodiments, the particles or plurality of particles, whether as a powder, compressed powder, or tablet, are spray coated. In some embodiments, the plurality of particles is a loose powder within an ingestible capsule. In some embodiments, the plurality of particles is compressed into a friable solid.

In some embodiments, the plurality of particles comprises one or more of:
1) a surface weighted mean (D3,2) of about 1.5-1.9 μm (e.g., about 1.7 μm); 2) a volume weighted mean diameter (D4,3) of about 3.5-4.5 μm (e.g., about 3.9 μm);
3) a d(0.1) of about 0.5-0.9 μm (e.g., about 0.7 μm);
4) a d(0.5) of about 2.5-3.5 μm (e.g., about 2.9 μm);
5) a d(0.9) of about 6-9 μm (e.g., about 7.8 μm);
6) a span of about 0.7-3.4 (e.g., about 2.4);
7) a uniformity coefficient of about 0.6-1.0 (e.g., about 0.8); or
8) a refractive index of about 1.4-1.6 (e.g., about 1.5).

In some embodiments, provided herein are oral dosage forms, comprising a particle, a composition, or a pharmaceutical composition described herein. In some embodiments, the oral dosage form comprises a plurality of the particles as a powder or as a compressed powder.

In some embodiments, the particles, compositions, or pharmaceutical compositions provided herein are housed in at least one container.

### Compositions

In some embodiments, the particles described herein may be in the form of a composition. In some embodiments, the composition is a pharmaceutical composition further comprising a pharmaceutically acceptable carrier.

In some embodiments, the compositions described herein comprise a first pharmaceutical active, which is Compound 1 or a salt thereof, and a second pharmaceutical active, which may be a compound useful in treating a disease or disorder of the central nervous system.

### Methods

Compound 1, and pharmaceutically acceptable salts thereof, may be used as described in U.S. Pat. Nos. 8,003,646, 8,399,467, or 8,921,366. Compound 1, or a pharmaceutically acceptable salt thereof, is described therein as a GABA_{A} receptor modulator, and useful in treating disorders of the central nervous system, including anxiety, convulsions, neuropathic pain, inflammatory pain, and migraine-associated pain.

Additionally, therapeutic uses of a corresponding non-deuterated form of Compound 1 or other GABA_{A} receptor modulators, or a pharmaceutically acceptable salt thereof, are described in U.S. Pat. Nos. 6,255,305 or 6,500,828, or WO2006061428, the entire content of each of which are incorporated by reference. Uses described in U.S. Pat. No. 6,255,305 include the treatment of a variety of disorders of the central nervous system, such as: anxiety disorders, such as panic disorder with or without agoraphobia; agoraphobia without history of panic disorder; animal and other phobias including social phobias; obsessive-compulsive disorder; stress disorders including post-traumatic and acute stress disorder and generalized or substance-induced anxiety disorder; neuroses; convulsions; migraine; and depressive or bipolar disorders, for example single-episode or recurrent major depressive disorder, dysthymic disorder, bipolar I and bipolar II manic disorders, and cyclothymic disorder. Uses described in U.S. Pat. No. 6,500,828 include the treatment of a variety of disorders of the central nervous system, in addition to those described above, such as: psychotic disorders including schizophrenia; neurodegeneration arising from cerebral ischemia; attention deficit hyperactivity disorder; and disorders of circadian rhythm, e.g. in subjects suffering from the effects of jet lag or shift work. Furthermore, uses described in U.S. Pat. No. 6,500,828 include disorders for which selective ligands for GABA_{A} receptors may be of benefit such as: pain and nociception; emesis, including acute, delayed and anticipatory emesis, in particular emesis induced by chemotherapy or radiation, as well as post-operative nausea and vomiting; eating disorders, including anorexia nervosa and bulimia nervosa; premenstrual syndrome; muscle spasm or spasticity, e.g. in paraplegic patients; and hearing loss. Additionally, U.S. Pat. No. 6,500,828 states that selective ligands for GABA_{A} receptors may also be effective as pre-medication prior to anesthesia or minor procedures such as endoscopy, including gastric endoscopy. Uses described in WO2006061428 include treatment of pain, e.g., neuropathic, inflammatory, or migraine associated pain. WO2006061428 states that neuropathic pain encompasses a range of pain syndromes of diverse origins including diabetic neuropathy, post-herpetic neuralgia, nerve injuries after surgery, pain following paraplegia, hypersensitivity to non-painful stimuli (allodynia), e.g. after surgery or during migraine attacks, spontaneous pain, hyperalgesia, diffuse muscle tenderness of myofacial syndromes, sensory abnormalities of the gastrointestinal tract, e.g. in irritable bowel disease, or chest pain and a large proportion of back pain, and also states that cancer- and AIDS-associated pain also qualify as neuropathic pain. WO2006061428 also states that inflammatory pain encompasses pain associated with conditions such as trauma, osteoarthritis, rheumatoid arthritis, post-surgery recovery, and some forms of cancer pain.

Accordingly, in some embodiments, the particles described herein, which include Compound 1, or a pharmaceutically acceptable salt thereof, are useful in treating GABA_{A} receptor related diseases, diseases or disorders of the central nervous system, and diseases or disorders as described above. In some embodiments, the particles described herein may be useful as an anxiolytic, anticonvulsant, amnesic, sedative, hypnotic, euphoriant, or muscle relaxant.

In some embodiments, particles described herein are useful as GABA_{A} receptor modulators, and in treating various disorders or diseases of the central nervous system.

In some embodiments, particles described herein are useful as an anxiolytic or an analgesic. Thus, in some embodiments, described herein are methods of treating anxiety or pain in a subject in need thereof, comprising administering to the subject a therapeutically effective amount of a particle described herein.

In some embodiments, described herein are methods of treating GABA_{A} receptor related diseases or a disease or disorder of the central nervous system in a subject in need thereof, comprising administering to the subject a therapeutically effective amount of the particle, composition, pharmaceutical composition, or oral dosage form described herein.

While the methods as described refer to the particles described herein, it is to be understood that the particles may be used in conjunction with these methods in the form of a composition or a pharmaceutical composition as well.

Actual dosage levels of the active ingredients (e.g., the pharmaceutical active compound (e.g., Compound 1) of the particles described herein), the compositions, or the pharmaceutical compositions provided herein may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health, or prior medical history of the patient being treated.

Routes of administration of include, without limitation, oral, nasal, rectal, intravaginal, aerosol, parenteral, buccal, sublingual, ophthalmic, pulmonary, or topical administration. In some embodiments, the oral or nasal route of administration is an oral inhalational or nasal inhalational route of administration. The compounds for use as described herein may be formulated for administration by any suitable route to achieve the particular method being applied.

Accordingly, administration of a compound, a composition, or a combination disclosed herein includes a variety of enteral or parenteral approaches selected from, without limitation: oral administration in any acceptable form, such as, e.g., tablet, liquid (e.g., liquid suspension of particles), capsule, powder, or the like; topical or transdermal administration in any acceptable form, such as, e.g., drops, spray, creams, gels ointments, or patches; buccal, nasal, sublingual, ophthalmic, pulmonary, and/or inhalation administration in any acceptable form; rectal administration in any acceptable form; vaginal administration in any acceptable form; peri- and intra-tissue administration in any acceptable form, such as, e.g., intraperitoneal injection, intramuscular injection, subcutaneous injection, intravenous injection, or intraarticular injection; intravesicular administration in any acceptable form, such as, e.g., catheter instillation; and by placement device, such as, e.g., an implant, a stent, a patch, a pellet, a catheter, an osmotic pump, a suppository, a bioerodible delivery system, a non-bioerodible delivery system or another implanted extended or slow release system.

Local administration results in significantly more delivery of a compound, a composition, or a combination to a specific location as compared to the entire body of the mammal, whereas, systemic administration results in delivery of a compound, a composition, or a combination to essentially the entire body of the individual. Routes of administration suitable for or treating a central nervous system related disease or disorder as disclosed herein also include both central and peripheral administration. Central administration results in delivery of a compound, a composition, or a combination to essentially the central nervous system of the individual and includes, e.g., nasal administration, intrathecal administration, epidural administration as well as a cranial injection or implant. In some embodiments, central administration is used to administer the compound, composition, or combinations described herein.

Central administration by the nasal route, which targets drug absorption through the vascular plexus of the nasal cavity, is distinct from administration by nasal inhalation, which delivers drug through the pulmonary system. Whereas the latter typically uses liquid or dry powder aerosols with mean particle sizes less than about 10 microns, central administration may be accomplished using mean particle sizes of about 10 microns or larger. Mists and aerosols can be generated using nebulizers, dry powder inhalers, pressurized aerosols, and atomization pumps. It is also feasible to use nose drops (e.g., a suspension of particles in a liquid) for central administration by the nasal route.

Peripheral administration results in delivery of a compound, a composition, or a combination to essentially any area of an individual outside of the central nervous system and encompasses any route of administration other than direct administration to the spine or brain.

### Kits

In some embodiments, provided herein are packaged particles, packaged compositions, or packaged pharmaceutical compositions, comprising a container holding a therapeutically effective amount of a particle described herein, and instructions for using the particle in accordance with one or more of the methods provided herein.

The present particles and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g., Irgacure 184, 2959), preferably water-soluble initiators (e.g., Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g. by the addition of specific product information leaflets) into suitable containers (boxes, etc.).

According to further embodiments, the present particles can also be provided in kit form combined with other components necessary for administration of the material to the patient. For example, disclosed kits, such as for use in the treatment of cancer, can further comprise, for example, administration materials.

The kits may be designed in various forms based on the specific deficiencies they are designed to treat.

The particles or compositions provided herein may be prepared and placed in a container for storage at ambient or elevated temperature. When the particle or composition is stored in a polyolefin plastic container as compared to, for example, a polyvinyl chloride plastic container, discoloration of the particle (e.g., a compound in the particle) or composition may be reduced, whether suspended in a liquid composition (e.g., an aqueous or organic liquid solution), or as a solid. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some containers also include the capacity to reduce exposure of the container's contents to infrared light, or a second component with such a capacity. Some containers further include the capacity to reduce the exposure of the container's contents to heat or humidity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper container, cardboard container, paperboard container, metallic film container, or foil container, or a combination thereof, to further reduce exposure of the container's contents to UV, visible, or infrared light. Articles of manufacture benefiting from reduced discoloration, decomposition, or both during storage, include dosage forms that include a particle or composition described herein. The particles or compositions provided herein may need storage lasting up to, or longer than, three months; in some cases up to, or longer than one year. The containers may be in any form suitable to contain the contents-for example, a bag, a bottle, or a box.

The following examples further illustrate aspects of the present disclosure. However, they are in no way a limitation of the teachings or disclosure as described herein.

### EXAMPLES

### Example 1

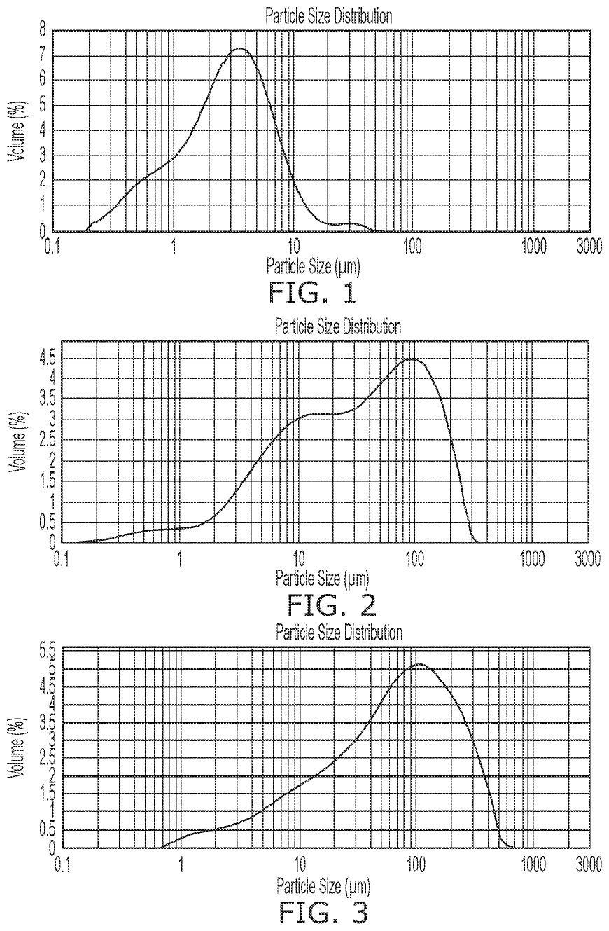

Micronized particles of Compound 1 free base were generated, and subsequently analyzed using a Mastersizer 2000 instrument and a Scirocco 2000 dispersion unit. Data of the particle analysis are shown in Table 1 and Table 2, as well as FIG. 1. Data of non-micronized particles (Examples 2a and 2b, below) used as comparators are shown in Tables 3 and 4, as well as FIG. 2 and FIG. 3.

TABLE 1
Micronized particle analysis of Example 1 (see FIG. 1).

| | | | |
|---|---|---|---|
| Particle Name: | Accessory Name: | Analysis model: | Sensitivity: |
| Example 1 | Scirocco 2000 | General purpose | Normal |
| Particle RI: | Absorption: | Residual: | Obscuration: |
| 1.520 | 0.1 | 3.072% | 2.40% |
| Dispersant Name: | Dispersant RI: | Weighted | Result |
| Not Applicable | 1.000 | Residual: | Emulation: |
| | | 0.447% | Off |
| Concentration: | Span: | Uniformity: | Result units: |
| 0.0001% Vol | 2.431 | 0.841 | Volume |
| Specific Surface | Surface Weighted | Volume Weighted | |
| Area: | Mean D[3, 2]: | Mean D[4, 3]: | |
| 3.62 m²/g | 1.656 μm | 3.948 μm | |
| d(0.1): | d(0.5): | d(0.9): | |
| 0.700 μm | 2.904 μm | 7.761 μm | |

TABLE 2
Micronized particle size distribution data
corresponding to Example 1 (see FIG. 1).

| Size (μm) | Volume (%) |
|---|---|
| 0.010-0.011 | 0.000 |
| 0.011-0.013 | 0.000 |
| 0.013-0.015 | 0.000 |
| 0.015-0.017 | 0.000 |
| 0.017-0.020 | 0.000 |
| 0.020-0.023 | 0.000 |
| 0.023-0.026 | 0.000 |
| 0.026-0.030 | 0.000 |
| 0.030-0.035 | 0.000 |
| 0.035-0.040 | 0.000 |
| 0.040-0.046 | 0.000 |
| 0.046-0.052 | 0.000 |
| 0.052-0.060 | 0.000 |
| 0.060-0.069 | 0.000 |
| 0.069-0.079 | 0.000 |
| 0.079-0.091 | 0.000 |
| 0.091-0.105 | 0.000 |
| 0.105-0.120 | 0.000 |
| 0.120-0.138 | 0.000 |
| 0.138-0.158 | 0.000 |
| 0.158-0.182 | 0.000 |
| 0.182-0.209 | 0.020 |
| 0.209-0.240 | 0.250 |
| 0.240-0.275 | 0.420 |
| 0.275-0.316 | 0.630 |
| 0.316-0.363 | 0.880 |
| 0.363-0.417 | 1.190 |
| 0.417-0.479 | 1.470 |
| 0.479-0.550 | 1.700 |
| 0.550-0.631 | 1.900 |
| 0.631-0.724 | 2.070 |
| 0.724-0.832 | 2.240 |
| 0.832-0.955 | 2.430 |
| 0.955-1.096 | 2.680 |
| 1.096-1.259 | 3.000 |
| 1.259-1.445 | 3.420 |
| 1.445-1.660 | 3.920 |
| 1.660-1.905 | 4.500 |
| 1.905-2.188 | 5.100 |
| 2.188-2.512 | 5.680 |
| 2.512-2.884 | 6.170 |
| 2.884-3.311 | 6.500 |
| 3.311-3.802 | 6.620 |
| 3.802-4.365 | 6.480 |
| 4.365-5.012 | 6.100 |
| 5.012-5.754 | 5.490 |
| 5.754-6.607 | 4.720 |
| 6.607-7.586 | 3.870 |
| 7.586-8.710 | 3.010 |
| 8.710-10.000 | 2.230 |
| 10.000-11.482 | 1.580 |
| 11.482-13.183 | 1.040 |
| 13.183-15.136 | 0.660 |
| 15.136-17.378 | 0.430 |
| 17.378-19.953 | 0.300 |
| 19.953-22.909 | 0.260 |
| 22.909-26.303 | 0.250 |
| 26.303-30.200 | 0.260 |
| 30.200-34.674 | 0.250 |
| 34.674-39.811 | 0.200 |
| 39.811-45.709 | 0.110 |
| 45.709-52.481 | 0.000 |
| 52.481-60.258 | 0.000 |
| 60.258-69.183 | 0.000 |
| 69.183-79.433 | 0.000 |
| 79.433-91.201 | 0.000 |
| 91.201-104.713 | 0.000 |
| 104.713-120.226 | 0.000 |
| 120.226-138.038 | 0.000 |
| 138.038-158.489 | 0.000 |
| 158.489-181.970 | 0.000 |
| 181.970-208.930 | 0.000 |
| 208.930-239.883 | 0.000 |
| 239.883-275.423 | 0.000 |
| 275.423-316.228 | 0.000 |
| 316.228-363.078 | 0.000 |
| 363.078-416.869 | 0.000 |
| 416.869-478.630 | 0.000 |
| 478.630-549.541 | 0.000 |
| 549.541-630.957 | 0.000 |
| 630.957-724.436 | 0.000 |
| 724.436-831.764 | 0.000 |
| 831.764-954.993 | 0.000 |
| 954.993-1096.478 | 0.000 |
| 1096.478-1258.925 | 0.000 |
| 1258.925-1445.440 | 0.000 |
| 1445.440-1659.587 | 0.000 |
| 1659.587-1905.461 | 0.000 |
| 1905.461-2187.762 | 0.000 |
| 2187.762-2511.886 | 0.000 |
| 2511.886-2884.032 | 0.000 |
| 2884.032-3311.311 | 0.000 |
| 3311.311-3801.894 | 0.000 |
| 3801.894-4365.158 | 0.000 |
| 4365.158-5011.872 | 0.000 |
| 5011.872-5754.399 | 0.000 |
| 5754.399-6606.934 | 0.000 |
| 6606.934-7585.776 | 0.000 |
| 7585.776-8709.636 | 0.000 |
| 8709.636-10000.000 | 0.000 |

### Example 2

Non-micronized particles of Compound 1 free base were generated, and subsequently analyzed using a Mastersizer 2000 instrument and a Scirocco 2000 dispersion unit. Data of the particle analysis are shown in Table 3 and Table 4, as well as FIG. 2 and FIG. 3.

TABLE 3
Non-micronized particle analysis of Example 2 (FIG. 2).

| | | | |
|---|---|---|---|
| Particle Name: | Accessory Name: | Analysis model: | Sensitivity: |
| Example 2a | Scirocco 2000 | General purpose | Normal |
| Particle RI: | Absorption: | Residual: | Obscuration: |
| 1.520 | 0.1 | 1.943% | 2.16% |
| Dispersant Name: | Dispersant RI: | Weighted | Result |
| Not Applicable | 1.000 | Residual: | Emulation: |
| | | 0.987% | Off |
| Concentration: | Span: | Uniformity: | Result units: |
| 0.0005% Vol | 4.330 | 1.34 | Volume |
| Specific Surface | Surface Weighted | Volume Weighted | |
| Area: | Mean D[3, 2]: | Mean D[4, 3]: | |
| 0.762 m²/g | 7.879 μm | 58.379 μm | |
| d(0.1): | d(0.5): | d(0.9): | |
| 4.166 μm | 34.165 μm | 152.108 μm | |

TABLE 4
Non-micronized particle analysis of Example 2 (FIG. 3).

| | | | |
|---|---|---|---|
| Particle Name: | Accessory Name: | Analysis model: | Sensitivity: |
| Example 2b | Scirocco 2000 | General purpose | Normal |
| Particle RI: | Absorption: | Residual: | Obscuration: |
| 1.520 | 0.1 | 0.997% | 2.41% |
| Dispersant Name: | Dispersant RI: | Weighted | Result |
| Not Applicable | 1.000 | Residual: | Emulation: |
| | | 0.936% | Off |
| Concentration: | Span: | Uniformity: | Result units: |
| 0.0013% Vol | 3.556 | 1.09 | Volume |
| Specific Surface | Surface Weighted | Volume Weighted | |
| Area: | Mean D[3, 2]: | Mean D[4, 3]: | |
| 0.329 m²/g | 18.264 μm | 104.115 μm | |
| d(0.1): | d(0.5): | d(0.9): | |
| 7.810 μm | 69.917 μm | 256.435 μm | |

### Example 3

Exposure of Compound 1 free base following oral (PO) dose administration of 300 mg/kg/dose of micronized particles (Example 1) and 300 mg/kg/dose non-micronized particles (Example 2) were assessed in male Cynomolgus monkeys. Table 5 describes the study design dosing and schedule. Blood samples were collected in K₂EDTA coated polypropylene tubes from the femoral vein and plasma was separated. Samples were drawn at the following time points: pre-dose, 15 min, 30 min, 1, 2, 4, 8, 10, 12, 14, 24, 30, and 48 hours post-dose. The concentrations of Compound 1 were determined by LC-MS/MS and pharmacokinetic parameters were determined using Phoenix WinNonlin v8.0) software. A summary of the pharmacokinetic parameters is shown in Table 6.

TABLE 5
Study design.

| | | | | Dose | Dose | |
|---|---|---|---|---|---|---|
| | | | Dose | Concentration | Volume | |
| Group | Test Article | N = (male) | (mg/kg) | (mg/mL) | (mL/kg) | Dosing |
| 1 | Micronized | 3 | 300 | 37.5 | 8 | QD |
| | Compound 1 | | | | | |

Animals were subjected to a 1-week wash-out period before transitioning to group 2.

| | | | | | | |
|---|---|---|---|---|---|---|
| 2 | Non-Micronized | 3 | 300 | 37.5 | 8 | QD |
| | Compound 1 | | | | | |

TABLE 6
Summary of pharmacokinetic parameters of Example 3.

| | Group 1 | Group 2 |
|---|---|---|
| | (QD, 300 mg/kg, | (QD, 300 mg/kg, |
| | micronized) | non-micronized) |
| Parameter | Mean (n = 3) | Mean (n = 3) |
| Tₘₐₓ (hr) | 4.00 ± 3.50 | 3.30 ± 1.20 |
| Cₘₐₓ (ng/mL) | 6900 ± 5498 | 5373 ± 4112 |
| AUCₗₐₛₜ (hr · ng/mL) | 158865 ± 160961 | 89225 ± 104664 |
| AUC_{∞} (hr · ng/mL) | 194908 ± 218989 | 98365 ± 119106 |
| AUCₗₐₛₜ/Dose (hr · kg · ng/mL/mg) | 530 ± 537 | 297 ± 349 |
| AUC_{∞}/Dose (hr · kg · ng/mL/mg) | 650 ± 730 | 328 ± 397 |

### Example 4

A pharmacokinetic analysis was performed comparing administration of Compound 1 with certain salts of Compound 1 after oral (PO) administration in 3 male cynomolgus monkeys. The subjects received a single PO oral gavage of active pharmaceutical ingredient (API) (e.g., Compound 1 fumarate, Compound 1 sulfate, Compound 1 hydrochloride, Compound 1 phosphate, Compound 1 tosylate, Compound 1 malonate, Compound 1 maleate, or Compound 1 free base) in a capsule at a dose of 30 mg/kg. Following dose administration, the animals were flushed with approximately 10 milliliters of tap water to ensure all API was administered. Animals were observed twice daily and at sample collection time-points for any abnormal clinical and behavioral signs. Body weights were taken prior to dosing and weekly thereafter until study termination. Blood samples (0.5 mL) were collected pre-dose, 15 minutes, 30 minutes, and 1, 2, 4, 6, 8, 10, 12, and 24 hours post-dose. Each blood sample was collected from the monkey's femoral, saphenous or other available vein via direct venipuncture, placed into a polypropylene tube containing K₂EDTA as the anticoagulant, and gently inverted several times to mix. The blood samples were kept on wet ice until centrifugation. Blood was centrifuged within 10 minutes of collection. The samples were centrifuged at a temperature of 4° C., at 3,000 g, for 5 minutes. The resulting plasma (˜0.25 mL) was divided into two equal aliquots (125 μL each) into polypropylene tubes, designated as Set A and Set B, after centrifugation. Plasma samples designated for certain metabolites (Set A) were treated with 12.5 μL of 2 M ascorbic acid (plasma: ascorbic acid=9:1, v:v) prepared beforehand by combining 0.352 g of ascorbic acid into 1 mL of sterile water in a glass vial and mixing thoroughly. Plasma samples designated for Compound 1 (Set B) do not require treatment with ascorbic acid. Once plasma was prepared, the samples were snap frozen on dry ice. All plasma samples were stored frozen below −70° C. until analyzed. Results are shown in Table 7.

Surprisingly, the fumarate salt of Compound 1 had a Cₘₐₓ of about 3080 ng/mL, which is over 98% higher than that of Compound 1 (free base; 1550 ng/mL). The fumarate salt of Compound 1 has an AUCₗₐₛₜ of 38904 ng-h/mL, which is over 50% higher than that of Compound 1 (25463 ng-h/mL).

TABLE 7
Pharmacokinetic data.

| | Free | Fumarate | Sulfate | Hydrochloride |
|---|---|---|---|---|
| Form | base | Salt | Salt | Salt |
| Cₘₐₓ (ng/mL) | 1550 | 3080 | 1151 | 1583 |
| Tₘₐₓ (h) | 4.0 | 4.7 | 4.0 | 4.0 |
| AUCₗₐₛₜ | 25463 | 38904 | 18231 | 25402 |
| (ng*h/mL) | | | | |
| | Phosphate | Tosylate | Malonate | Maleate |
| Form | Salt | Salt | Salt | Salt |
| Cₘₐₓ (ng/mL) | 1096 | 1629 | 2010 | 1388 |
| Tₘₐₓ (h) | 5.3 | 10.7 | 4.7 | 12.0 |
| AUCₗₐₛₜ | 18378 | 23248 | 24694 | 20575 |
| (ng*h/mL) | | | | |

### Example 5

A hemi-fumarate Form A (acetonitrile solvate) and hemi-fumarate Form B (non-solvated) of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine were prepared. A seed crystal for Form B was prepared by first combining 20.3 mg (0.050 mM) 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine with 1000 μL acetone. Fumaric acid/EtOH was added (248 mg, 1.0 eq) and the sample was continuously stirred while cycling the temperature between 40° C. and 5° C. (heating and cooling at 2° C./min. with 1 hour hold at 40° C. and 5° C.) for 48 hours. The samples were isolated at 5° C. and the suspension was equilibrated with stirring at 20° C. for 2 hours. Birefringent solids were harvested by filtration at room temperature and air-dried overnight. 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine (5.51 mmol) was combined with acetone (3.5 mL, 16 vol). To the resulting solution (with minimal residual solids), fumaric acid (575 mg, 1.0 eq) was added, followed by seeding, e.g., with Form B (˜10 mg). The slurry was heated to 40° C. for 2 h with stirring and cooled very slowly to 20° C. at 0.1° C./min rate and held for 1 h at every 2° C. interval and stirred at 20° C. overnight. The solids were isolated by vacuum filtration, air-dried for 4 h. The yield was 79% (2.02 g, 4.33 mmol) of hemi-fumarate salt Form B. Form A (prepared as above but with acetonitrile instead of acetone) converted to Form B at ambient conditions in one week.

Selected physicochemical data of Form B were collected. Hemi-fumarate salt Form B was determined to be crystalline by polarized-light microscopy (PLM) and XRPD. DSC analysis showed a very small endotherm at 194.3° C. (ΔH=1.7 J/g), followed immediately by a sharp endotherm at 199.6° C. (ΔH=105 J/g), and TGA analysis showed 0.7% wt loss up to 190° C. (see FIG. 7). Proton NMR confirmed a hemi-salt with 0.5:1 CI:API ratio. Gravimetric vapor sorption (GVS) analysis showed <0.1% wt moisture uptake between 5-95% RH. No change in the XRPD pattern was observed post GVS.

The intrinsic dissolution rate was determined by forming compacted pellets of approximately 75 mg of API using a compression force of 1200 pounds of force, and each pellet (die surface area: 0.5 cm²) was immersed into a flat bottom vessel containing 500 mL of medium (aqueous 0.1 N HCl with 1% Tween 80 (polyoxyethylene (20) sorbitan monooleate)), using a Distek 2100A dissolution bath, and Distek Circulator/heater (set to 37° C.), with a paddle speed of 100 rpm. An aliquot of the sample (1 mL) was pulled from dissolution media at 15, 30, 45, 60, 75, 90, 105, and 120 minutes, filtered, and analyzed by HPLC. The slope of the dissolution profile corresponded to an intrinsic dissolution rate of hemi-fumarate salt Form B of 6.2 μg/(min·cm²) as compared to 8.9 μg/(min·cm²) for Compound 1 (free base).

Selected physicochemical data of Form A were collected. Hemi-fumarate salt Form A was determined to be crystalline by polarized-light microscopy (PLM) and XRPD. DSC analysis showed a sharp endotherm at 62.1° C. (ΔH=76 J/g), followed by a sharp endotherm at 195.2° C. (ΔH=109 J/g), and TGA analysis showed 7% wt loss up to 80° C. (see FIG. 6).

For PLM, photomicrographs were collected using Olympus BX60 polarized-light microscope equipped with Olympus DP70 camera or Olympus BX51 polarized-light microscope equipped with Olympus DP71 camera.

Solid-state stability of the fumarate salt of Compound 1 was assessed after storage at the following conditions for 2 and 4 weeks by HPLC and XRPD: 40° C./75% RH (opened); and 50° C./ambient RH (closed). No significant changes were observed by either HPLC or XRPD after 4 weeks under these conditions.

DSC was conducted with a TA Instruments Q200 or Q2000 differential scanning calorimeter equipped with an autosampler and a refrigerated cooling system under 40 mL/min N₂ purge. DSC thermograms of samples were obtained at 15° C./min in crimped Al pans, unless noted otherwise.

TGA thermograms were obtained with a TA Instruments Q500 thermogravimetric analyzer under 40 mL/min N₂ purge for balance and 60 mL/min for sample in Al pans. TGA thermograms of samples were obtained at 15° C./min, unless noted otherwise.

GVS experiments were conducted on a Surface Measurement Systems DVS-Advantage. The experiments were performed at 25° C. The instrument was operated in step mode and the relative humidity was increased in 10% RH increments from 40% RH to 75% RH, then decreased from 75% RH to 5% RH, then increased a second time from 5% RH to 95% RH, then decreased from 95% RH to 5% RH. The mass equilibrium criterion was set at 0.003% change in mass over time (dm/dt). A minimum step time of 20 minutes and a maximum step time of 240 minutes were specified.

### Example 6

Powder X-ray diffraction patterns were collected for selected salt forms described herein, including hemi-fumarate Form A (acetonitrile solvate) and hemi-fumarate Form B (non-solvated) of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine (see FIG. 4 and FIG. 5, respectively). XRPD diffractograms were acquired on a Bruker D8 Advance system using Cu Kα (λ1=1.54060 Å; λ2=1.54439 Å) radiation, X-ray tube voltage and current 40 kV and 40 mA, and a LynxEye position sensitive detector with a 3° 20 opening. The configuration on the incident beam side was as follows: Göebel mirror, mirror exit slit (0.2 mm), 2.5 deg Soller slits, and beam knife. The configuration on the diffracted beam side was as follows: anti-scatter slit (8 mm) and 2.5 deg Soller slits. Samples were mounted flat on zero-background Si wafers. Samples were rotated at 30 rpm. Data was collected at a step size of 0.029° 2θ and step time of 0.1 second. Data was collected from 2 to 40° 2θ for a total scan time of 2 minutes 22 seconds per sample. XRPD patterns were imported into Panalytical HighScore Plus v 2.2. The Kα2 contribution was mathematically stripped from the patterns followed by determining the background baseline followed by determining signal positions and relative intensities. Alternatively, XRPD diffractograms were acquired on PANalytical X'Pert Pro diffractometer using Ni-filtered Cu Kα (45 kV/40 mA) radiation and a step size of 0.030 20 and X'celerator RTMS (Real Time Multi-Strip) detector. Configuration on the incidental beam side: variable divergence slits (10 mm irradiated length), 0.04 rad Soller slits, fixed anti-scatter slit (0.50°), and 10 mm beam mask. Configuration on the diffracted beam side: variable anti-scatter slit (10 mm observed length) and 0.04 rad Soller slits. Samples were mounted flat on zero-background Si wafers. Results are shown in Table 8.

TABLE 8
XRPD signals for hemi-fumarate Form A
and hemi-fumarate Form B of Example 5.

| | | |
|---|---|---|
| 2θ [deg] | d-spacing [Å] | Rel. Int. [%] |

hemi-fumarate Form A

| | | |
|---|---|---|
| 7.70 | 11.5 | 62 |
| 10.61 | 8.33 | 14 |
| 11.20 | 7.89 | 11 |
| 15.30 | 5.79 | 43 |
| 15.62 | 5.67 | 34 |
| 20.20 | 4.39 | 21 |
| 21.58 | 4.11 | 65 |
| 21.80 | 4.07 | 100 |
| 22.56 | 3.94 | 23 |
| 25.44 | 3.50 | 20 |
| 28.75 | 3.10 | 33 |
| 30.73 | 2.91 | 16 |

hemi-fumarate Form B

| | | |
|---|---|---|
| 8.60 | 10.3 | 7 |
| 9.05 | 9.76 | 100 |
| 10.01 | 8.83 | 3 |
| 11.03 | 8.01 | 5 |
| 11.83 | 7.47 | 3 |
| 12.95 | 6.83 | 8 |
| 15.46 | 5.73 | 23 |
| 16.63 | 5.33 | 6 |
| 17.29 | 5.13 | 8 |
| 19.20 | 4.62 | 11 |
| 20.14 | 4.41 | 5 |
| 20.55 | 4.32 | 13 |
| 21.83 | 4.07 | 13 |
| 22.46 | 3.96 | 33 |
| 24.25 | 3.67 | 10 |
| 24.61 | 3.61 | 16 |
| 29.89 | 2.99 | 5 |

### Example 7. Oral Non-Human Primate Crossover Study

An objective of this study is to determine the Pharmacokinetic (PK) profile of the Test Article following a single oral administration to four male non-human primate cynomolgus monkeys with one (1) week wash out periods. Dosing and crossover design is as shown in Table 9.

TABLE 9

| Dose Group/ | | Dose Level | | Capsule Per | Blood Sampling |
|---|---|---|---|---|---|
| No. Animals | Test Article | (PO Capsule) | | Animal | Time Points |
| G1/N = 4 | Micronized fumarate | 34.2 | mg/kg* | 1 capsule on Day | Predose, 0.25, 0.5, |

| | | |
|---|---|---|
| salt of Compound 1 | 1 immediately | 1, 2, 4, 8, 10, 12, |
| (M-3) | followed by 10 | 14, and 24 hours |
| | mL H₂O oral flush | post-dose |

1 week washout period after last time point of blood collection

| | | | | | |
|---|---|---|---|---|---|
| G2/N = 4 | Non-micronized | 34.2 | mg/kg* | 1 capsule on Day | Predose, 0.25, 0.5, |

| | | |
|---|---|---|
| fumarate salt of | 1 immediately | 1, 2, 4, 8, 10, 12, |
| Compound 1 (N-3) | followed by 10 | 14, and 24 hours |
| | mL H₂O oral flush | post-dose |

1 week washout period after last time point of blood collection

| | | | | | |
|---|---|---|---|---|---|
| G3/N = 4 | Non-micronized | 30 | mg/kg | 1 capsule on Day | Predose, 0.25, 0.5, |

| | | |
|---|---|---|
| Compound 1 as a | 1 immediately | 1, 2, 4, 8, 10, 12, |
| free base (K-13) | followed by 10 | 14, and 24 hours |
| | mL H₂O oral flush | post-dose |

Animals return to colony
*34.2 mg/kg fumarate salt of Compound 1 is equivalent on a molar basis to 30 mg/kg free base of Compound 1.

Four male animals are used for this study, and the four study animals are used for each of three dosing phases with one-week washout periods. Food is withheld overnight prior to dose administration, and food is returned four hours post-dose administration. Water is provided ad libitum by automatic watering device. Two of the four animals are previously treated with Compound 1, and the other two are Compound 1 naive. Test Article (M-3, N-3, or K-13) are provided as capsulized powders; stored at room temperature protected from light in, for example, a polyolefin bottle. Table 10 summarizes the study results, which show that the fumarate salt of Compound 1 in both micronized and non-micronized form provided superior exposure to Compound 1 as compared to subjects dosed with the free base of Compound 1.

TABLE 10

| Parameter | Units | M-3 | N-3 | K-13 |
|---|---|---|---|---|
| AUC | (ng · h/mL) | 17826 | 21540 | 1192 |
| Cₘₐₓ | (ng · mL) | 930 | 1370 | 86.4 |
| Tₘₐₓ | (h) | 10 | 4 | 14 |
| C₂₄₍ₕ₎ | (ng · mL) | 603 | 684 | 42 |

Micronized and non-micronized particles of Compound 1 hemi-fumarate in this example are described in Table 11 (micronized) and Table 12 (non-micronized).

TABLE 11
Micronized Compound 1 Hemi-Fumarate

| | | | | |
|---|---|---|---|---|
| X₁₀ = 1.41 μm | X₅₀ = 13.74 μm | X₉₀ = 42.98 μm | SMD = 4.48 μm | VMD = 19.16 μm |
| X₈₀ = 30.52 μm | X₉₈ = 82.27 μm | X₁₀₀ = 174.00 μm | Sᵥ = 1.34 m²/cm³ | Sₘ = 13399.69 cm²/g |

TABLE 12
Non-Micronized Compound 1 Hemi-Fumarate

| | | | | |
|---|---|---|---|---|
| X₁₀ = 0.40 μm | X₅₀ = 1.00 μm | X₉₀ = 2.14 μm | SMD = 0.80 μm | VMD = 1.19 μm |
| X₈₀ = 1.67 μm | X₉₈ = 3.81 μm | X₁₀₀ = 6.25 μm | Sᵥ = 7.46 m²/cm³ | Sₘ = 74582.13 cm²/g |

### Example 8. Intrinsic Dissolution Rate

Determination of intrinsic dissolution rate (IDR) was performed for Compound 1 and seven different salts of Compound 1 according to USP <1087> in 0.1 N hydrochloric acid with 1% Tween 80 (Polyoxyethylene (80) sorbitan monooleate). Compacted pellets containing approximately 75 mg API were prepared using a compression force of 1200 pounds of force, and each pellet (die surface area: 0.5 cm²) was immersed into a flat bottom vessel containing 500 mL of medium, using a Distek 2100A dissolution bath, and Distek Circulator/heater (set to 37° C.), with a paddle speed of 100 rpm. An aliquot of the sample (1 mL) was pulled from dissolution media at 15, 30, 45, 60, 75, 90, 105, and 120 minutes, filtered, and analyzed by HPLC.

A summary of the intrinsic dissolution rate and solubility of each compound tested is shown in Table 13 and Table 14.

TABLE 13
Solubility

| Time Point (min) | 15 | 30 | 45 | 60 | 75 | 90 | 105 | 120 | Cumulative |
|---|---|---|---|---|---|---|---|---|---|
| Compound 1 | 0.413 | 0.464 | 0.641 | 0.823 | 0.89 | 1.026 | 1.23 | 1.31 | 6.797 |
| (parent free | | | | | | | | | |
| base) (μg/mL) | | | | | | | | | |
| Fumarate | 0.342 | 0.404 | 0.46 | 0.576 | 0.684 | 0.79 | 0.87 | 0.993 | 5.119 |
| Salt (μg/mL) | | | | | | | | | |
| Hydrochloride | 0.452 | 0.358 | 0.561 | 0.59 | 0.723 | 0.847 | 0.989 | 1.13 | 5.65 |
| Salt (μg/mL) | | | | | | | | | |
| Maleate Salt | 0.245 | 0.36 | 0.462 | 0.567 | 0.681 | 0.791 | 0.941 | 1.01 | 5.057 |
| (μg/mL) | | | | | | | | | |
| Malonate Salt | 0.352 | 0.454 | 0.579 | 0.68 | 0.757 | 0.87 | 1.04 | 1.05 | 5.782 |
| (μg/mL) | | | | | | | | | |
| Phosphate | 0.358 | 0.375 | 0.449 | 0.543 | 0.615 | 0.769 | 0.912 | 0.11 | 4.131 |
| Salt (μg/mL) | | | | | | | | | |
| Sulfate Salt | 0.316 | 0.364 | 0.453 | 0.569 | 0.668 | 0.772 | 0.944 | 1.06 | 5.146 |
| (μg/mL) | | | | | | | | | |
| Tosylate Salt | 0.56 | 0.638 | 0.755 | 0.88 | 1.02 | 1.15 | 1.43 | 1.39 | 7.823 |
| (μg/mL) | | | | | | | | | |

TABLE 14
Intrinsic Dissolution Rate

| | IDR [μg/(min · cm²)] |
|---|---|
| Compound 1 (parent free base) | 8.9 |
| Fumarate Salt | 6.2 |
| HCl Salt | 6.9 |
| Maleate Salt | 7.3 |
| Malonate Salt | 6.9 |
| Phosphate Salt | 7.0 |
| Sulfate Salt | 7.1 |
| Tosylate Salt | 8.7 |

### Example 9. Solid-State Stability

Solid-state stability of Compound 1 and seven salts of Compound 1, as in Example 8, was assessed after storage at the following conditions for 2 and 4 weeks by HPLC and PXRD: 40° C./75% RH (opened); and 50° C./ambient RH (closed).

No significant changes were observed in HPLC data of compounds exposed to 40° C./75% RH (opened), and 50° C./ambient RH (closed) for up to 4 weeks. XRPD analysis of the stability samples showed no significant change in SRPD patterns after 4 weeks in the storage conditions, except in the HCl salt with observation of a small signal at 10° 2θ of Compound 1 (free base) at 40° C./75% RH.

HPLC for IDR and solid-state stability analyses were conducted with an Agilent 1260 Infinity system equipped with a G1311B Quad pump, G1329B Autosampler, G1330B auto-sampler thermostat, G1316A Thermostatted Column Compartment with a column switch valve, and G4212B diode array detector.

### Example 10. Melting Point

Melting points of Compound 1 and seven of its salts (as in Example 8) were collected, which results are summarized in Table 15.

TABLE 15
Melting Points

| | Temperature (° C.) |
|---|---|
| Compound 1 (parent free base) | 202 |
| Fumarate Salt (Form B) | 199 |
| HCl Salt (Form A) | 190 |
| Maleate Salt (Form A) | 171 |
| Malonate Salt (Form A) | 173 |
| Phosphate Salt (Form A) | 217 |
| Phosphate Salt (Form B) | 228 |
| Sulfate Salt (Form A) | 228 |
| Tosylate Salt (Form A) | 227 |

We claim:

1. A compound, which is a fumarate salt of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine.

2. The compound of claim 1, which is a hemi-fumarate salt of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine.

3. The compound of claim 1, which is of the formula: 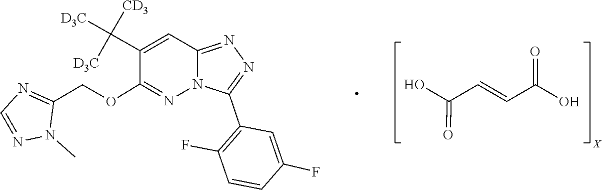
wherein X is 0.5, 1, or 2.

4. The compound of one of claims 1-3, which is an anhydrate, hemihydrate, monohydrate, or dihydrate.

5. The compound of one of claims 1-3, which is a solvate.

6. The compound of one of claims 1-5, which is in a solid form.

7. The compound of claim 6, wherein the solid form has an X-ray powder diffraction pattern comprising a 2-theta signal, based on CuKα1 radiation (1.54060 Å), at about 9.05 ±0.2°, or based on d-spacing, at about 9.76 Å.

8. The compound of claim 6, wherein the solid form has an X-ray powder diffraction pattern comprising 2-theta signals, based on CuKα1 radiation (1.54060 Å), at about 9.05±0.2°, about 15.46±0.2°, about 22.55±0.2°, and about 24.61±0.2°, or based on d-spacing, at about 9.76 Å, about 5.73 Å, about 3.96 Å, and about 3.61 Å.

9. The compound of claim 6, wherein the solid form has an X-ray powder diffraction pattern substantially as shown in FIG. 5.

10. The compound of claim 6, wherein the solid form has a differential scanning calorimetry thermogram comprising an endothermic signal at about 199.6±3.0 (e.g., +0.5) ° C.

11. The compound of claim 6, wherein the solid form has a differential scanning calorimetry thermogram substantially as shown in FIG. 7.

12. The compound of claim 6, wherein the solid form has a thermogravimetric analysis substantially as shown in FIG. 7.

13. The compound of claim 6, wherein the solid form has an X-ray powder diffraction pattern comprising a 2-theta signal based on CuKα1 radiation (1.54060 Å) at about 7.70±0.2° or about 21.80±0.2°, or based on d-spacing at about 11.5 Å or about 4.07 Å.

14. The compound of claim 6, wherein the solid form has an X-ray powder diffraction pattern comprising 2-theta signals, based on CuKα1 radiation (1.54060 Å), at about 7.70±0.2°, about 21.80±0.2°, about 25.44±0.2°, and about 28.75±0.2°, or based on d-spacing at about 11.5 Å, about 4.07 Å, about 3.50 Å, and about 3.10 Å.

15. The compound of claim 6, wherein the solid form has an X-ray powder diffraction pattern substantially as shown in FIG. 4.

16. The compound of claim 6, wherein the solid form has a differential scanning calorimetry thermogram comprising an endothermic signal at about 62.1±3.0 (e.g., ±0.5)° C., about 195.2±3.0 (e.g., ±0.5)° C., or both.

17. The compound of claim 6, wherein the solid form has a differential scanning calorimetry thermogram substantially as shown in FIG. 6.

18. The compound of claim 6, wherein the solid form has a thermogravimetric analysis substantially as shown in FIG. 6.

19. The compound of one of claims 1-18, which is substantially purified.

20. The compound of one of claims 1-19, which is crystalline.

21. The compound of one of claims 1-20, prepared by a process comprising precipitating the compound from a solution comprising 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine, fumaric acid, and a solvent comprising acetone or acetonitrile, the process optionally comprising drying the precipitated compound.

22. A composition, comprising the compound of one of claims 1-21.

23. The composition of claim 22, which is a pharmaceutical composition further comprising a pharmaceutically acceptable carrier.

24. The composition of claim 23, wherein the compound is present in the composition in an amount of at least about 90% by weight.

25. The composition of claim 22, which is a pharmaceutical composition consisting essentially of the compound.

26. A particle, comprising 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine, or a pharmaceutically acceptable salt thereof, or the composition of one of claims 22-25, wherein the particle comprises one or more of:
1) a specific surface area of about 3.0-4.0 m²/g (e.g., about 3.6); or
2) a diameter of about 0.15-50 μm.

27. The particle of claim 26, wherein the compound is about, or at least about, 75, 80, 85, 90, 95, or 100% by mass of the particle.

28. The particle of claim 26 or 27, comprising a particle surface wherein the particle surface comprises a coating on at least a portion of the particle surface.

29. The particle of claim 26 or 27, wherein the particle is encapsulated within a coating.

30. The particle of claim 28 or 29, wherein the coating is 25% or less by mass of the coated particle.

31. The particle of one of claims 26-30, wherein the particle comprises a diameter of about 0.2-20 μm (e.g., about 1-10 μm, e.g., about 2-6 μm, e.g., about 4 μm).

32. The particle of one of claims 26-31, wherein the particle is a solid particle.

33. A composition, comprising the particle of one of claims 26-32.

34. A composition, comprising a plurality of particles of one of claims 26-32.

35. The composition of claim 34, wherein the plurality of particles comprises one or more of:
1) a surface weighted mean (D3,2) of about 1.5-1.9 μm (e.g., about 1.7 μm);
2) a volume weighted mean diameter (D4,3) of about 3.5-4.5 μm (e.g., about 3.9 μm);
3) a d(0.1) of about 0.5-0.9 μm (e.g., about 0.7 μm);
4) a d(0.5) of about 2.5-3.5 μm (e.g., about 2.9 μm);
5) a d(0.9) of about 6-9 μm (e.g., about 7.8 μm);
6) a span of about 0.7-3.4 (e.g., about 2.4);
7) a uniformity coefficient of about 0.6-1.0 (e.g., about 0.8); or
8) a refractive index of about 1.4-1.6 (e.g., about 1.5).

36. A pharmaceutical composition, comprising a pharmaceutically acceptable carrier, and the particle of one of claims 26-32 or the composition of one of claims 33-35.

37. An oral dosage form, comprising the particle of one of claims 26-32, the composition of one of claims 33-35, or the pharmaceutical composition of claim 36.

38. The oral dosage form of claim 37, wherein the oral dosage form comprises a plurality of the particles as a powder or as a compressed powder.

39. The compound of one of claims 1-21, the composition of one of claims 22-25, the particle of one of claims 26-32, the composition of one of claims 33-35, the pharmaceutical composition of claim 36, or the oral dosage form of claim 37 or 38, housed in at least one container.

40. A method of treating GABA_{A} receptor related diseases or a disease or disorder of the central nervous system in a subject in need thereof, comprising administering to the subject a therapeutically effective amount of the compound, particle, composition, pharmaceutical composition, or oral dosage form of one of claims 1-39.

41. A compound, which is a sulfate, hydrochloride, phosphate, tosylate, malonate, or maleate salt of 3-(2,5-difluorophenyl)-7-[1,1-di(methyl-d₃)ethyl-2,2,2-d₃]-6-[(1-methyl-1H-1,2,4-triazol-5-yl)methoxy]-1,2,4-triazolo[4,3-b]pyridazine.

42. A method, comprising administering to a subject the compound, particle, composition, pharmaceutical composition, or oral dosage form of one of claims 1-39 or the compound of claim 41.
